(19) United States
(12) Patent Application Publication
(10) Pub. No.: US 2025/0000037 A1
(43) Pub. Date: Jan. 2, 2025

(54) Production and use of the leaf and/or leaves and/or stem(s) and/or stalk(s) of plants of the cannabis species for consumption as a food, beverage ingredient, supplement, medicine

(71) Applicant: david alan heldreth, JR., bellevue, WA (US)

(72) Inventor: david alan heldreth, JR., bellevue, WA (US)

(21) Appl. No.: 18/882,639

(22) Filed: Sep. 11, 2024

Related U.S. Application Data
(62) Division of application No. 17/689,706, filed on Mar. 8, 2022.

(51) Int. Cl. A01G 22/15; A01C 7/00; A01D 45/06; A23L 2/52; A23L 29/00; A24B 15/16; A24B 15/167; A61K 36/185; C12G 3/04

(52) U.S. Cl. CPC A01G 22/15; A01C 7/00; A01D 45/065; A23L 2/52; A23L 29/00; A24B 15/16; A24B 15/167; A61K 36/185; C12G 3/04; A61K 2236/13; A61K 2236/15

(57) ABSTRACT

This present invention is directed to a method for the production and use of the leaf and/or leaves and/or stem(s) and/or stalk(s) of plants of the cannabis species for animal, microbial, bacteria, fungi or human consumption as a food, supplement, medicine, beverage.

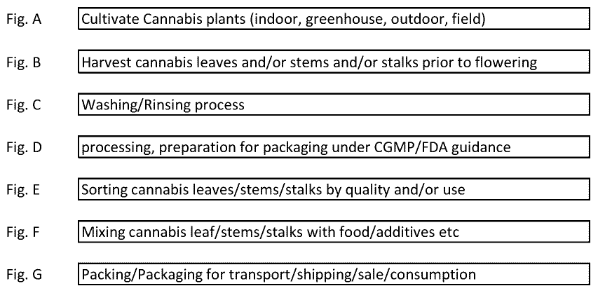

### FIELD OF THE INVENTION

This present invention is directed to a method for the production and use of the leaf and/or leaves and/or stem(s) and/or stalk(s) of plants of the cannabis species for animal, microbial, bacteria, fungi or human consumption as a food, supplement, medicine.

### BACKGROUND OF THE INVENTION

The cannabis plant has been cultivated for thousands of years for a variety of uses from the seed for food use as a seed oil, raw seed, extracted seed protein. It has also been used as a medicine by taking the cannabinoids and other compounds out of the plant. However, there has been little to no use of the whole plant parts of the cannabis plant, especially beyond the cannabis flower. The primary use previously described for the cannabis leaf and stalk are for juicing, however this method requires heavy processing and does separates the whole plant parts into different components. The whole plant parts contain a synergy of nutrition and medical value which is not accessible through separation and reconstitution.

As such we now present a complete system of production and use of the leaf and/or leaves and/or stem(s) and/or stalk(s) of plants of the cannabis species prior to flowering.

### SUMMARY OF THE INVENTION

This present invention is directed to a method for the production and use of the leaf and/or leaves and/or stem(s) and/or stalk(s) of plants of the cannabis species prior to flowering for animal, microbial, bacteria, fungi or human consumption as a food, beverage ingredients, supplement, medicine.

### BRIEF DESCRIPTION OF THE DRAWINGS

The drawing shows one embodiment of the invention in which the cannabis follows this process flow from:

FIG. A: Cultivating cannabis; to

FIG. B: harvesting cannabis leaf and/or leaves and/or stem(s) and/or stalk(s) prior to flowering; to

FIG. C: rinsing and/or chilling and/or washing and/or the cannabis leaf and/or leaves and/or stem(s) and/or stalk(s) either during harvest/within 12 hours of harvest; to

FIG. D: further preparing the cannabis leaf and/or leaves and/or stem(s) and/or stalk(s) for packaging according to current good manufacturing processes (CGMP) and/or FDA guidance; to

FIG. E: sorting the cannabis leaf and/or leaves and/or stem(s) and/or stalk(s) into plant parts and/or by quality and/or by intended use;

FIG. F: Mixing cannabis leaf and/or leaves and/or stem(s) and/or stalk(s) with any single item or a mixture of items from this list: food ingredients, food additives, encapsulation, beta glucan particles, chitosan, yeast extract, surfactants, binders and other compounds to increase efficiency, availability, release lifespan, release speed among other parameters; and finally to FIG. G: packing/packaging for transport and/or shipping and/or manufacturing and/or sale and/or consumption.

### DRAWING SUMMARY

The drawing shows one embodiment of the invention in which the cannabis follows this process flow from:

FIG. A: Cultivating cannabis; to

FIG. B: harvesting cannabis leaf and/or leaves and/or stem(s) and/or stalk(s) prior to flowering; to

FIG. C: rinsing and/or chilling and/or washing and/or the cannabis leaf and/or leaves and/or stem(s) and/or stalk(s) either during harvest/within 12 hours of harvest; to

FIG. D: further preparing the cannabis leaf and/or leaves and/or stem(s) and/or stalk(s) for packaging according to current good manufacturing processes (CGMP) and/or FDA guidance; to

FIG. E: sorting the cannabis leaf and/or leaves and/or stem(s) and/or stalk(s) into plant parts and/or by quality and/or by intended use;

FIG. F: Mixing cannabis leaf and/or leaves and/or stem(s) and/or stalk(s) with any single item or a mixture of items from this list: food ingredients, food additives, encapsulation, beta glucan particles, chitosan, yeast extract, surfactants, binders and other compounds to increase efficiency, availability, release lifespan, release speed among other parameters; and finally to

FIG. G: packing/packaging for transport and/or shipping and/or manufacturing and/or sale and/or consumption.

### DETAILED DESCRIPTION OF THE INVENTION

This present invention is directed to a method for the production and use of the leaf and/or leaves and/or stem(s) and/or stalk(s) of plants of the cannabis species for animal, microbial, bacteria, fungi or human consumption as a food, supplement, medicine.

In one embodiment the method includes the any mixture of the following steps: (a) harvesting cannabis leaf and/or leaves and/or stem(s) and/or stalk(s) prior to flowering; (b) rinsing and/or chilling and/or washing and/or the cannabis leaf and/or leaves and/or stem(s) and/or stalk(s) either during harvest/within 12 hours of harvest; (c) further preparing the cannabis leaf and/or leaves and/or stem(s) and/or stalk(s) for packaging according to current good manufacturing processes (CGMP) and FDA guidance; (d) sorting the cannabis leaf and/or leaves and/or stem(s) and/or stalk(s) into plant parts and/or by quality and/or by intended use; and (e) packing/packaging for transport and/or shipping and/or sale and/or consumption.

In one embodiment the cannabis leaf and/or leaves and/or stem(s) and/or stalk(s) are organically grown. In one embodiment the cannabis leaf and/or leaves and/or stem(s) and/or stalk(s) are grown indoors. In one embodiment the cannabis leaf and/or leaves and/or stem(s) and/or stalk(s) are grown outdoors. In one embodiment the cannabis leaf and/or leaves and/or stem(s) and/or stalk(s) are grown in a greenhouse. In one embodiment the cannabis leaf and/or leaves and/or stem(s) and/or stalk(s) are grown in a field. In one embodiment the cannabis leaf and/or leaves and/or stem(s) and/or stalk(s) are grown hydroponically. In one embodiment the cannabis leaf and/or leaves and/or stem(s) and/or stalk(s) are grown aeroponically. In one embodiment the cannabis leaf and/or leaves and/or stem(s) and/or stalk(s) are grown with conventional nutrients.

In one embodiment, the cannabis leaf and/or leaves and/or stem(s) and/or stalk(s) are harvested simultaneously from the same plant to reduce harvest time, labor, cost and potential contamination from individually collecting leaf and/or leaves, stem(s) or stalk(s) from plants. In one embodiment the cannabis leaf and/or leaves and/or stem(s) and/or stalk(s) are harvested utilizing mechanical harvesting equipment such as, but not limited to combines or spinach/microgreen/spring mix harvesters such as made by companies: Ramsay, Tumoba, Ortomec.

In one embodiment the cannabis leaf and/or leaves and/or stem(s) and/or stalk(s) are used as whole plants. In one embodiment the cannabis leaf and/or leaves and/or stem(s) and/or stalk(s) are ground to be mixed into foods. In one embodiment the cannabis leaf and/or leaves and/or stem(s) and/or stalk(s) are separated after harvest. In one embodiment the cannabis leaf and/or leaves and/or stem(s) and/or stalk(s) are sorted utilizing artificial intelligence and/or visual/infrared/density and other scanners.

In some embodiment the cannabis leaf and/or leaves and/or stem(s) and/or stalk(s) are consumed raw. In some embodiment the cannabis leaf and/or leaves and/or stem(s) and/or stalk(s) are cooked. In some embodiment the cannabis leaf and/or leaves and/or stem(s) and/or stalk(s) are frozen. In some embodiments the cannabis leaf and/or leaves and/or stem(s) and/or stalk(s) are dehydrated. In some embodiment the cannabis leaf and/or leaves and/or stem(s) and/or stalk(s) are used individually.

In some embodiments the cannabis contains below 0.3% total delta-9-tetrahydrocannabinol (D9THC) content as calculated as Total D9THC=(0.877*% D9THC-acid)+% D9THC. In some embodiments the cannabis contains below 0.3% total (THC) in which this calculation includes a total of all tetrahydrocannabinol isomers and analogs such as, but not limited to delta-8-tetrahydrocannabinol, delta-10-tetrahydrocannabinol, 11-hydroxy-tetrahydrocannabinol as required by the FDA, DEA or other government agency.

Some embodiments will include utilizing nano technology, encapsulation, beta glucan particles, chitosan, yeast extract, surfactants, binders and other compounds to increase efficiency, availability, release lifespan, release speed among other parameters.

In some embodiments cannabis leaf and/or leaves and/or stem(s) and/or stalk(s) are used in foods, supplements, inhalation products such as, but not limited to: vaporizers, cigarettes, cigars, infused smoking papers, infused smoking blends, cigarillos, vape fluids, eye drops, nasal spray, mouth spray, rectal inhalers or other uses.

Some embodiments are used to develop vaccines or treatments for virus, fungi or bacteria illness such as, but not limited to: Coronavirus (such as SARS, COVID-19, MERS), HIV, herpes, syphilis, flu virus, swine flu, avian flu, other virus, bacteria or fungi: antibodies-cytokins-proteins-amino acids-DNA-RNA-DNA/RNA to produce antibodies/proteins/amino acids/RNA/DNA; such as for the spike or other proteins used in vaccines or treatment of virus, fungi or bacteria illness.

1. A method for preparing a packaged cannabis leaf food product consisting of:
a. cultivating cannabis or hemp that has more than 7% wt. % protein in the whole cannabis leaf;
b. harvesting the cannabis prior to flowering;
c. rinsing, chilling and/or washing the cannabis either during the harvest or within 12 hours of the harvest;
d. sorting the cannabis leaf/leaves from the rest of the cannabis;
e. mixing the cannabis leaves with a mixture of:
salt, pepper, rosemary, dill, mustard seeds, coriander, bell pepper(s), cucumber(s) (pickled or not), olive(s), and carrot(s), into an acidic solution (below ph 4.6); OR salt, pepper, sugar, garlic, mustard seeds, all-spice, coriander, bell pepper(s), cucumber(s)/pickles, olive(s), and carrot(s), into an acidic solution (below ph 4.6); OR salt, pepper, sugar, red pepper flakes, pepper sauce, garlic, mustard seeds, all-spice, coriander, cucumber(s)/pickles, and hot peppers, into an acidic solution (below ph 4.6); AND
f. packaging the cannabis leaf food product in an acidic solution for transportation or sale such as in a jar, bottle, bag, box, container, or otherwise.

2. A method of claim 1 in which the blend(s) include additional items such as, but not limited to: salt, pepper, cilantro, basil, oregano, rosemary, clove, mustard, dill, fennel, cannabis/hemp flower(s), cucumber(s), carrot(s), olive(s), caper(s), tomato(es), beet(s), olive oil, pepper(s) or other fruits/vegetables, onions, mustard seed(s), hemp seed(s), cumin, tumeric, vanilla, cinnamon, coffee, tea, sesame seed, fruit(s), vegetable(s), spices, essential oils, pimento, cabbage, horse radish, wasabi, vinegar, soy sauce, rice wine vinegar, flavors.

3. The method of claim 1, wherein the cannabis leaf food product or ingredients are disinfected via UV or irradiation or or frozen or pasteurized or cooked.

4. The method of claim 1, further consists of a preservative(s) selected from the group consisting of sodium benzoate, potassium sorbate, sulfur dioxide, rosemary oil, vitamin e and vitamin c.

5. A method for preparing a pre-packaged cannabis leaf food product consisting of:
a. cultivating cannabis which has more than 7% wt. % protein in the entire cannabis leaf;
b. harvesting the cannabis prior to flowering;
c. rinsing, chilling and/or washing the cannabis either during the harvest or within 12 hours of the harvest;
d. sorting the cannabis leaf/leaves from the rest of the cannabis;
e. mixing the cannabis leaves/leaf with a mixture of: soy sauce, pepper flakes, miso paste, rice wine vinegar, sugar, garlic, brown sugar, green onion (powder or otherwise); OR hot sauce, pepper flakes, sugar, brown sugar, hot pepper(s), mustard, oregano, onion; and
f. packaging the cannabis leaf food product with for transportation and/or for sale and/or consumption such as in a jar, bottle, bag, box, container, or otherwise.

6. A method of claim 5 in which the cannabis leaf food item is dehydrated prior to packaging.

7. A method of claim 5 in which the cannabis leaf food item is fried with or without coating (such as, but not limited to breading or spice blend) prior to packaging.

8. A method of claim 5 in which the blend(s) include additional items such as, but not limited to: salt, pepper, cilantro, basil, oregano, rosemary, clove, mustard, dill, fennel, cannabis/hemp flower(s), cucumber(s), carrot(s), olive(s), caper(s), tomato(es), beet(s), olive oil, pepper(s) or other fruits/vegetables, onions, mustard seed(s), hemp seed(s), cumin, tumeric, sesame seed, fruit(s), vegetable(s), spices, essential oils, pimento, flavors, meat(s), cheese(s), cumin, wasabi, horse radish, pepper(s), tumeric, vanilla, cinnamon, coffee, tea, or cannabis extract(s).

9. The method of claim 5, wherein the cannabis leaf food product or ingredients are disinfected via UV or irradiation or or frozen or pasteurized or cooked.

10. The method of claim 5, further consists of a preservative(s) selected from the group consisting of sodium benzoate, potassium sorbate, sulfur dioxide, rosemary oil, vitamin e and vitamin c.

11. A method for producing cannabis leaf and/or leaves, stem(s) and stalk(s) for use in items such as, but not limited to: food(s), beverage(s), alcoholic beverage(s), supplement(s), inhalation products (cigarette, vapor products, e-cigarettes, cigars, pre rolls, joints, pipe mixes, hookah), recreational products or medicine(s) consisting of any mixture of the following steps:
a) Cultivating cannabis
b) Harvesting cannabis leaf and/or leaves and/or stem(s) and/or stalk(s) prior to flowering;
c) rinsing and/or chilling and/or washing and/or the cannabis leaf and/or leaves and/or stem(s) and/or stalk(s) either during harvest/within 12 hours of harvest;
d) further preparing the cannabis leaf and/or leaves and/or stem(s) and/or stalk(s) for packaging according to current good manufacturing processes (CGMP) and/or FDA guidance;
e) sorting the cannabis leaf and/or leaves and/or stem(s) and/or stalk(s) into plant parts and/or by quality and/or by intended use;
f) Mixing cannabis leaf and/or leaves and/or stem(s) and/or stalk(s) with any single item or a mixture of items from this list: food ingredients, food additives, supplements, medicines, preservatives, essential oils, flavors, nano technology, encapsulation, beta glucan particles, chitosan, yeast extract, surfactants, binders and other compounds to increase efficiency, availability, release lifespan, release speed among other parameters; AND
g) Packing/packaging for transport and/or shipping and/or manufacturing and/or sale and/or consumption.

12. The method of claim 11 in which the cannabis contains below 0.3% THC.

13. The method of claim 11 in which the final cannabis product contains below 0.3% THC.
